(19) United States
(12) Patent Application Publication
(10) Pub. No.: US 2007/0026075 A1
(43) Pub. Date: Feb. 1, 2007

(54) Gelled donepezil compositions and methods for making and using the same

(76) Inventors: Jutaro Shudo, San Jose, CA (US); Kunio Yoneto, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/476,410

(22) Filed: Jun. 27, 2006

Related U.S. Application Data
(60) Provisional application No. 60/704,104, filed on Jul. 28, 2005.

(51) Int. Cl. A61K 31/445; A61K 9/14

(52) U.S. Cl. 424/488; 514/319

(57) ABSTRACT

Gelled donepezil compositions and methods for making and using the same are provided. The subject compositions include a donepezil active agent in a gelled oral pharmaceutically acceptable vehicle comprising an emulsion of water and oil. Also provided are kits of the subject compositions.

### CROSS-REFERENCE TO RELATED APPLICATIONS

Pursuant to 35 U.S.C. § 119 (e), this application claims priority to the filing dates of: U.S. Provisional Patent Application Ser. No. 60/704,104 filed Jul. 28, 2005; the disclosures of which is herein incorporated by reference.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

It has been known that senile dementia such as Alzheimer-type senile dementia, cerebrovascular dementia, attention deficit hyperactivity disorder and the like are accompanied by a reduction in cholinergic functions in the brain. At present, it has been recognized that acetylcholinesterase inhibitors are effective as an agent for treating these diseases.

Donepezil hydrochloride ((i)-2,3-dihydro-5,6-dimethoxy-2-[[1-(phenylmethyl)4-piperidinyl]methyl]-1H-inden-1-one hydrochloride) is now marketed under the trademark Aricept®, and has been in widespread use in the treatment of dementia associated with Alzheimer's and related conditions. Donepezil hydrochloride, which is the hydrochloride salt of donepezil (CAS#:120014-064) is commonly referred to in the pharmacological literature as E2020, has an empirical formula of C₂₄H₂₉NO₃HCl and a molecular weight of 415.96. The structural formula of Donepezil hydrochloride is: 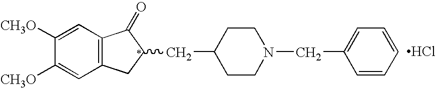
Donepezil hydrochloride is currently available in tablets, rapid disintegration tablets and liquid dosage formulations.

The target patients of donepezil hydrochloride include those patients suffering from senile dementia and Alzheimer's disease, whose ability to swallow declines. Also they frequently have the dry mouth symptoms due to aging. Because of their poor cognitive function, it often happens that the target patient population does not remember if they have taken the medicine, particularly when small tablet dosage forms are being employed. The general problem is that the solid dosage form is hard to swallow and the liquid dosage form can be ingested incorrectly with adverse consequences, e.g., choking and wrong way administration. As such, currently employed dosage formulations are not ideal for a significant portion of the target patient population. Therefore, the ideal dosage form for this medicine that is taken on a daily basis is such that patients can take with ease. For such a dosage form, a gelled composition for oral administration that has the properties of viscoelasticity and smooth texture is appropriate. However, a gelled composition for oral administration that contains donepezil hydrochloride has never been considered. In addition, since donepezil hydrochloride has an unpleasant taste such as bitterness and a tongue-biting sensation, a gelled composition for oral administration that can mask such a taste is desired.

References of Interest

Published patent literature of interest includes: U.S. Pat. Nos. 6,576,677, 6,252,081, 6,245,911, 6,193,993, 5,932,235, 4,895,841, 5,985,864, 6,140,321, US2004/0002517, US2004/0002517, US2004/0214863, JP 2004-217639, JP H11-228450, JP 2001-270821, JP H11-106354, JP H11-106353, JP H11-228450, JP H11-106354, JP 2005-15486, JP 2005-29541, JP 2005-41887, JP 2005-112825, JP 2005-126374, JP 2005-130791, JP 2005-132803, JP 2005-194230, JP 2005-194231, JP 2004-000496, JP 2004-10552, JP 200443450, JP 2004-73035, JP 2004-83523, JP 2004-174202, JP 2004-189706, JP 2004-217639, JP 2004-292316, JP 2004-292383, JP 2004-321249, JP 2004-359597, JP 2003-12507, JP 2003-26683, JP 2003-89631, JP 2003-137773, JP 2003-210553, JP 2003-277255, JP 2003-284739, JP 2002-179558, JP 2002-193807, JP 2002-193911, JP 2002-255796, JP 2002-255819, JP 2002-255820, JP 2002-322085, JP 2002-326934, JP 2001-139547, JP 2001-270821, JP 2001-302497, JP 2001-342151, JP 2000-136134, JP 2000-136183, JP 2000-143627, JP 2000-319257, JP 2000-319258, JP 2000-327590, JP H11-92402, JP H11-106353, JP H11-106354, JP H11-171861, JP H11-228450, JP H11-263774, JP H11-315016, JP H10-53576, JP H10-120553, PCT/JP99/04616 published as WO/2000/012135, PCT/JP01/05714 published as WO/2002/002526, W02001/064190, WO2004/002402, WO2002/026709, WO 2003/000261, WO2003/006013, WO2003/006423, WO2002/100399, WO2003/006453, WO2003/035137, WO2003/020370, WO2003/030886, WO2003/050073, WO2002/034915, WO2003/027068, WO2003/043987, WO2003/045378, WO2003/047576, WO2003/072535, WO2003/043618, WO2003/040096, WO2002/002506, WO2002/034237, WO2002/096415, WO2002/100818, WO2002/100856, WO2002/094768, WO2002/100820, WO2004/006021, WO2001/066114, WO2001/070672, WO1999/65489, WO1998/34615, WO2002/02526, WO2002/24167, WO2002/26709, WO2002/45715, WO2002/69942, WO2002/96370, WO2002/100384, WO2003/32960, WO2003/61658, WO2003/66100, WO2000/48580, WO2000/54811, WO2001/64190, WO2001/78728, WO2001/98271, WO1999/29668, WO2004/092137, WO2004/087660, WO2004/016589, WO2004/000317, and WO2002/26709.

Publications of interest include: Mukaiet al., “Clinical Efficacy of Acyclovir 800 mg Oral Administration of Jelly Type Drug on Herpes Zoster—Phase III Open Study,”JPN Pharmacol. Ther (2001) 29:347-361; Sakashita et al., “Clinical usefulness of jelly preparation , new formulation for medicinal drug,” Pharm. Tech. Japan (2003) 19:157-167; Dairaku & Togashi, “Development of Air Push Jelly Formulation and packaging Design,”JPI Journal (2003) 41:26-31; Dairaku, “Development of Air Push Jelly Formulation and It's packaging Design,”Convertech. (2003) 366:126-127; Dairaku & Togashi, “Oral Jelly Formulation For Ciloslet (TN) 50 mg and 100 mg (Generic Name, Cilostazol), Antiplatelat Aggregation Agent,” Journal of Neuro-Internal Medicine in Latest Available Medicine 30 (Technology and Academic Study Reference, 2004); and Dairaku & Togashi, “Development of Air-Push-Type-Jelly Formulation,” Journal of pharmaceutical science and technology. Japan (2005) 65:209-214.

### SUMMARY OF THE INVENTION

Gelled Donepezil compositions and methods for making and using the same are provided. The subject compositions include a donepezil active agent, e.g., donepezil hydrochloride, in a gelled oral pharmaceutically acceptable vehicle comprising an emulsion of water and oil. Also provided are kits of the subject compositions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

### DESCRIPTION OF REPRESENTATIVE EMBODIMENTS

The inventors of the present invention diligently carried out a research with the problem described above in mind and succeeded in making a gelled composition for oral administration. The subject compositions include a donepezil active agent, e.g., donepezil hydrochloride, in a gelled oral pharmaceutically acceptable vehicle comprising an emulsion of oil and water, e.g., a water-in-oil emulsion or an oil-in-water emulsion. One embodiment of the composition was made by adding donepezil, gelatinizing agents, thickening agents, sweetening agents and perfume to an emulsion of oil and water and adjusting pH between 3 and 7, which can be easy to swallow with a smooth texture for patients without experiencing an unpleasant taste such as bitterness and a sensation of tongue-biting and which has an effect of raising the patients visual awareness and attention by turning a clear water-soluble gelled composition of donepezil into a white emulsion. So, this invention can improve patient's quality of life. Accordingly, gelled donepezil compositions and methods for making and using the same are provided. Also provided are kits of the subject compositions.

In further describing the subject invention, the subject gel compositions and representative methods for their fabrication are described first in greater detail, followed by a review of representative methods and applications in which the gel compositions find use, as well as a review of representative kits that include the subject gel compositions.

Gelled Donepezil Compositions

As summarized above, the subject invention is directed to gelled compositions that include a donepezil active agent, e.g., donepezil or a salt thereof, such as donepezil hydrochloride. By “gel composition”. is meant a colloid in a more solid form than a sol. The strength of the subject compositions may vary, but in representative embodiments ranges from about 20 g/0.8 cm² to about 400 g/0.8 cm², such as from about 30 g/0.8 cm² to about 350 g/0.8 cm², including from about 40 g/0.8 cm² to about 300 g/0.8 cm².

A feature of the subject gel compositions is that the compositions are storage stable. By storage stable is meant that the compositions may be stored for extended periods of time without significant phase separation and/or significant reduction in activity of the donepezil active agent. In representative embodiments, the subject compositions are stable for at least about 2 months, such as at least about 4 months, including at least about 6 months or longer, e.g., at least about 1 year, at least about 1.5 years, etc., when maintained at 25° C. and 60% humidity. By the phrase “without substantially decreasing the activity of the donepezil active agent” is meant that at the end of the storage period, there is less than about 5% reduction in activity of the donepezil active agent compared to the beginning of the storage period.

A feature of the subject gelled compositions is that they have a pH below that at which donepezil is adversely effected, where the pH is generally less than about 8.0, and in representative embodiments ranges from about 3.0 to about 7.0, such as from about 3.5 to 6.0, and including from about 4.5 to about 5.5.

As mentioned above, the subject compositions include a donepezil active agent. The term “donepezil active agent” includes donepezil (CAS#:120014-064) or salts there, e.g., donepezil hydrochloride. The donepezil active agent may be purchased from commercial sources and produced using any of a variety of known synthesis protocols. Representative synthesis protocols including those synthesis protocols described in or referenced by U.S. Pat. Nos. 6,252,081; 6,245,911; 6,193,993; 6,140,321; 5,985,864; and 4,895,841 the disclosures of which are herein incorporated by reference. The amount of donepezil active agent that is present in a given composition is an amount sufficient to administer to a subject an effective amount of the agent when orally delivered to the subject. In certain embodiments, the amount of active agent present may range from about 0.001% to about 10.0% (w/w) or more (e.g., about 15.0%), e.g., from about 0.05% to about 10.0% (w/w), e.g., from about 0.05% to about 5.0% (w/w), e.g., from about 0.05% to about 3.0% (w/w) of the gel composition. In certain embodiments, the active agent is present in an amount ranging from about 0.0025 wt % to about 0.2 wt %.

A feature of the subject donepezil gel compositions is that the active agent is present in a gelled pharmaceutically acceptable vehicle that includes an emulsion of water and oil. The term emulsion is employed here in its conventional sense to refer to a mixture of two immiscible (unblendable) fluids, where one fluid (an oil or water) (the dispersed phase) is dispersed in the other fluid (an oil or water) (the continuous phase).

As the subject compositions include an emulsion of oil and water, they include water that may be present in an amount that ranges, in certain embodiments, from about 30% to about 70%, such as from about 40% to about 60%. The water may be any convenient water, include deinionized water, water for injection, etc., as is known in the art.

The oil component of the subject compositions may be any convenient orally acceptable oil or oils. Representative oils that may be employed include, but are not limited to: animal/vegetable oils, hardened animal/vegetable oils, fractionated animal/vegetable oils, fatty acid esters (medium-chain fatty acid glyceride, isopropyl myristate, octadecyl myristate), tricaprin, cacao oil, cacao butter, orange oil, soybean phospholipids, palm oil, peanut oil, soybean oil, cottonseed oil, soybean oil mixture, eucalyptus oil, lavender oil, lemon oil, rose oil, mint oil, fennel oil. As is known in the art, one or more of the above oils may be present, where the total oil content of the formulation, in representative embodiments, ranges from about 0.1 to 45 wt %, such as from about 0.5 to 30 wt %, including from about 1 to 20 wt %.

The subject compositions also typically include a surfactant. Any convenient orally acceptable surfactant or combination of surfactants may be present in the subject compositions.

Representative surfactants of interest include, but are not limited to: potassium palmitate, potassium lauryl sulfate, sodium lauryl sulfate, polyoxyethylene alkylethers, polyoxyethylene alkenylethers, polyoxyethylene alkylphenylethers, polyoxyethylene hydrogenated castor oil, polyoxyethylene glycerin fatty acid ester, polyoxyethylene sorbitan fatty acid ester, propylene glycol fatty acid ester, polyglycerin condensed ricinolein acid ester, lecithin, polyoxyethylene polyoxy propylene glycol, polyglycerin fatty acid ester (the olive oil HLB(Hydrophilic-Lipophilic Balance) value is about 10 or lower), sucrose fatty acid ester (the olive oil HLB value is about 10 or lower) and the like. In representative embodiments, the amount of surfactant ranges from about 0.05 to about 25 wt %, such as from about 0.1 to about 20 wt %, including from about 0.5 to about 10 wt %.

Compositions of the invention may also include a gelling agent. Representative gelling agents of interest include, but are not limited to: carrageenan, pectin, agar, alginic acid, sodium alginate, gelatin, mannan, konjak, konjak mannan, glucosic mannan, chitosan, acacia gum, xanthan gum, locust bean gum, tamarind seed polysaccharides, gellan gum, tragacanth gum, karaya gum, cassia gum, tara gum, guar gum, psyllium seed gum, gutti gum, pullulan, low methoxyl pectin, kudzu, casein, tapioca starch, alpha starch. In representative embodiments, the amount of gelling agent present in the composition ranges from about 0.01 to about 10 wt % (e.g., from about 0.01 to about 5.0 wt %); where ranges interest include from about 0.5 to about 10 wt %, such as from about 1 to about 7 wt %, and including from about 2 to about 5 wt %.

In representative embodiments, e.g., where locust bean gum is employed as a thickening agent (as reviewed in greater detail below), the gelling agent is a carrageenan or pectin. In those embodiments where the gelling agent includes a carrageenan, the carrageenan may be kappa or iota carrageenan (or combination thereof), but in representative embodiments is not lambda carrageenan. The total content of carrageenan, e.g., kappa type or iota type, in the composition of the present invention may range from about 0.5 to about 10 wt % to the total quantity, such as from about 1 to about 7 wt %, and including from about 2 to about 5 wt %.

Embodiments of the subject compositions also include a thickening agent. Any convenient thickening agent may be present, where representative thickening agents of interest include, but are not limited to: acacia gum, tragacanth gum, locust bean gum, dextrin, dextran, arabinogalactan, milk solid, pullulan, alginate propylene glycol ester, hydroxyethyl starch, carboxymethyl starch, hydroxypropyl starch copolydone, polyvinyl alcohol completely saponified, polyvinyl alcohol partially saponified, polyvinyl pyrrolidone, carboxyvinyl polymer, sodium polyacrylate, polyethylene glycolmacrogol, and cellulose or its derivatives such as cellulose gum, carmellose sodium, hydroxypropyl cellulose, hydroxyethyl methyl cellulose, methyl cellulose, carboxymethyl cellulose and copolyvidone, polyvinyl pyrrolidone, carboxyvinyl polymer, polyacrylic acid or partially neutralized polyacrylic acid, or salt and macrogol, etc.

In representative embodiments, the amount of thickening agent present in the composition ranges from about 0.01 to about 10 wt % (e.g., from about 0.01 to about 5.0 wt %); where ranges interest include from about 0.5 to about 10 wt %, such as from about 1 to about 7 wt %, and including from about 2 to about 5 wt %. Among the thickening agents described above, locust bean gum, a galactomannan polysaccharide which is extracted from carob, is employed in representative embodiments. Of particular interest is the use of locust bean gum when the gelling agent is carrageenan and/or pectin.

As reviewed above, the pH of the subject formulations is generally less than about 8.0, and in representative embodiments ranges from about 3.0 to about 7.0, such as from about 3.5 to 6.0, and including from about 4.5 to about 5.5. While any convenient buffering agent(s) may be employed to provide the desired pH, of interest in representative embodiments are cationic inorganic acids, cationic organic acids, or their salts. The additive percentage of cation inorganic acid or organic acid salts may range from about 0.1 to about 3%, such as from about 0.2 to about 2.5%, and including from about 0.3 to about 2%.

In representative embodiments, the gelling agents contained in the composition of the present invention such as pectin, alginic acid, alginate sodium, mannan, glucosic mannan, carrageenan, xanthan gum, tamarind seed polysaccharides, gellan gum, karaya gum, cassia gum, tara gum, guar gum, psyllium seed gum, and gutti gum become gelled more quickly by coexisting with metal ions such as potassium and calcium and do not separate from water as easily, where the overall effect of the buffering agent is to adjust the texture of the composition when it is taken orally. In this case, cation inorganic acid or organic acid salts to the total additive quantity of the gelatinizing agents may range from about 1 to about 600 wt %, such as from about 1.5 to about 300 wt %, and including from about 2 to about 150 wt %.

As described above, the gelled composition of the present invention can contain polyols, sweetening agents, flavoring agent and preservatives, e.g., as desired to adjust the taste, the smell, the texture and/or the ease of swallowing the composition by the subject.

Polyols of interest include, but are not limited to: glycerin, propylene glycol, D-sorbitol, xylitol, mannitol, erythritol and sucrose.

Sweetening agents of interest include but are not limited to: saccharine or glycyrrhiza, as well as sodium, potassium and ammonium salts thereof, e.g., saccharine sodium, dipotassium glycyrrhizinate, etc., which extraordinarily improves bitterness and a paralyzing sensation in an aqueous suspension, as well as aspartame, D-sorbitol, xylitol, mannitol, erythritol and sucrose. The blending concentration of these sweetening agents is, in representative embodiments, about 0.01 wt % or more, such as about 0.025 wt % or more. Furthermore, in order to make the taste a little milder, fructose, a proper quantity of refined sucrose, palatinose, trehalose, oligosaccharide, aspartame, isomerized sugar, muscovado, saccharine, saccharin sodium, sweet hydrangea leaf, powdered sweet hydrangea leaf, steviocide, licorice, licorice extract, glucose, starch syrup, maltose syrup powder, reduced malt sugar starch syrup, Kanbaiko may be present.

Any convenient or desirable flavoring agent may be present, where representative flavoring agents of interest include, but are not limited to: fennel, fennel oil, orange, orange extract, orange essence, orange oil, mint water, mint oil, honey, d-balneol, dl-menthol, l-menthol, eucalyptus oil, lavender oil, lemon oil, rose oil, sugar flavor, vanilla flavor, vanillin, chocolate flavorA22736, fruit flavor, cherry flavor, ethyl vanillin and various types of fruit juice. The quantity of the flavoring agent can be small.

Any convenient preservative may be present, where representative preservatives include, but are not limited to: sodium benzoate, edentate sodium, sodium salicylate, sorbic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, methyl parahydroxybenzoate, chlorobutanol, benzyl alcohol, phenylethyl alcohol and phenoxyethanol. The quantity of the preservative added may be readily determined based on the conventional quantity range for oral administration, as is known in the art.

The subject compositions can be produced using any convenient protocol. For example, the compositions of the present invention can be manufactured by adding the donepezil active agent, gelling agents, thickening agents, sweetening agents and flavorants to an emulsion of oil and water and ensuring that the pH is adjusted to the desired range, e.g., between about 3 and about 7.

In certain embodiments, the subject gelled compositions are present in single dosage form. In such embodiments, the amount of composition that makes up the single dosage form may vary, but in representative embodiments ranges from about 0.5 g to about 40 g, such as from about 1 g to about 30 g, including from about 2 g to about 20 g.

Where desired, the single dosage formulations of these embodiments may be packaged, e.g., in disposable packaging, e.g., for self-medication. In certain embodiments, the single dosage formulations are present in a sealed pouch, e.g., made of a polymeric material, (for example a sealed pouch having a “pillow” configuration) where the pouch may include a quantity of gas to assist in delivery of the formulation from the pouch to the subject upon use (described in Patent:JP2000-256181). Another convenient disposable packaging format that may be employed is the format disclosed in U.S. Pat. No. 5,932,235, the disclosure of which is herein incorporated by reference. Briefly, the packaging format includes a disposable container, e.g., which is made of a synthetic resin, composed of a shell being deformable under forcing and capable of containing the gelled composition for oral administration therein, a ring neck with a small diameter connected to the shell, and a handle serving as the closed end of the neck and connected to the neck in a breakable manner, wherein the neck is opened to provide a spout for pouring the gelled medical composition for oral administration when the handle is broken away from the neck.

Method of Using Gelled Donepezil Compositions

The subject gel compositions find use in the oral administration of donepezil to a subject. By “oral administration” is meant delivery via the mouth of the subject. In using the subject gel composition to orally administer donepezil to a subject, a dosage of the donepezil gel composition is introduced into the mouth of the subject and ingested. Where the dosage is present in a packaging, such as the disposable packaging described above, the packaging is opened and the dosage formulation present therein is removed and introduced into the mouth for ingestion, e.g., by swallowing.

Utility

The above described donepezil gel compositions and methods find use in any application in which the administration of donepezil to a subject is desired. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In certain embodiments, the subject methods find use in the treatment of a disease condition. By treatment is meant at least an amelioration of the symptoms associated with the pathological condition afflicting the subject, where amelioration is used in a broad sense to refer to at least a reduction in the magnitude of a parameter, e.g. symptom, associated with the pathological condition being treated, such as viral load or side effects associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition. As such, treatment includes both curing and managing a disease condition.

Representative disease conditions in which the subject compositions find use include, but are not limited to, disease conditions in which acetyl-cholinesterase inhibition is desired. As such, the subject compositions find use in the treatment of dementia, such as senile dementia, cerebrovascular dementia or attention deficit hyperactivity disorder. In particular, the subject compositions find use in the treatment of Alzheimer type senile dementia.

Kits

Also provided are kits, where the subject kits at least include a donepezil gel composition, as described above. The subject gel composition in the kits may be present in a package, as described above. Kits may include the donepezil gel composition in an amount suitable for a single application or multiple applications. In instances in which gel composition is present in a kit in an amount sufficient for more than one application, multiple packages, as described above, may be provided with each containing an amount of gel composition for a single application.

The subject kits may also include instructions for how to use the compositions in methods of donepezil delivery to a subject. The instructions may include information about dosing schedules etc., and/or how to use packaged gel compositions. In certain embodiments, the subject kits include instructions on how to use the gel compositions to treat a particular disease condition, e.g., dementia. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following practical and comparative examples are offered by way of illustration and not by way of limitation.

### EXAMPLES

The present invention will be described in more details below, referring to practical examples, but the scope of the present invention is not limited thereby.

Practical Example

The necessary amounts of purified water (K) and glycerin (D) are placed in a preparation tank, and components A, C, F, H and I are dissolved in the solution of water and glycerin while being heated. Next, components B are added and stirred and emulsify. Then, components E and G added under stirring to the emulsion at 80 to 90° C. so that they are dissolved and dispersed in the emulsion. Component J is added and dispersed, and the resultant composition is sterilized with heat. A disposable container is filled with a unit dose of 3 g or 6 g and chilled to make a product and individually packed unit dosage of the gelled donepezil formulation.

| Ingredient | | Ex. 1-1 | Ex. 1-2 | Ex. 1-3 | Ex. 1-4 | Ex. 1-5 | Ex. 1-6 |
|---|---|---|---|---|---|---|---|
| A | Donepezil hydrochloride | 0.1 | 0.1 | 0.1 | 0.1 | 0.1 | 0.1 |
| B | Orange oil | | 0.45 | | 5 | | |
| | Soybean oil | | | | | 5 | |
| | Medium-chain fatty acid glyceride | 5 | 10 | 5 | | | 5 |
| C | Sucrose fatty acid ester | 0.175 | 0.355 | 0.2 | 0.2 | 0.2 | 0.2 |
| | Polyglycerin fatty acid ester | | | 0.2 | 0.2 | 0.2 | 0.2 |
| | Glycerin fatty acid ester | | 0.845 | | | | |
| D | Glycerin | 13.5 | 13.5 | 10 | 15 | 15 | 15 |
| E | I-carrageenan | 0.45 | 0.45 | 0.5 | 0.5 | 0.5 | 0.5 |
| | K-carrageenan | 0.3 | 0.2 | 0.2 | | | |
| | Locust bean gum | 0.1 | 0.2 | 0.2 | 0.2 | 0.2 | 0.2 |
| | Xanthan gum | 0.05 | | | | | |
| | Sodium polyacrylate | 0.003 | 0.003 | 0.003 | 0.03 | 0.03 | |
| | Pectin | | | | 0.2 | 0.2 | 0.2 |
| | Agar | | | | 0.1 | | |
| F | Propyl parahydroxybenzoate | 0.015 | 0.015 | 0.015 | 0.015 | 0.015 | 0.015 |
| G | Saccharine sodium | | | | 0.1 | 0.1 | 0.1 |
| | Dipotassium glycyrrhizinate | | 0.2 | | | | |
| | D-sorbitol | 30 | 30 | 30 | 30 | 30 | 30 |
| H | Citric acid | | | | 0.1 | 0.1 | 0.1 |
| I | Sodium citrate | | | | Proper | Proper | Proper |
| J | Flavor | 0.1 | | 0.1 | | 0.1 | 0.1 |
| K | Purified water | Proper | Proper | Proper | Proper | Proper | Proper |
| | Total quantity | 100 | 100 | 100 | 100 | 100 | 100 |

Unit: g.

### Taste Test

Test Formulations

The necessary amount of purified water (K) and glycerin (D) are placed in a preparation tank, and components A, C and F are dissolved in the solution of water and glycerin while being heated. Next, components B are added and stirred and emulsify. Then, components E and G added under stirring to the emulsion at 80 to 90° C. so that they are dissolved and dispersed in the emulsion. Component J is added and dispersed, and the resultant composition is sterilized with heat. A disposable container is filled with a unit dose of 3 g and chilled to make a product and individually packed unit dosage of the gelled donepezil formulation.

| | | | T-02 | |
|---|---|---|---|---|
| | | | (Com- | T-03 |
| | | | parative | (Comparative |
| Ingredient | | T-01 | example) | Example) |
| A | Donepezil hydrochloride | 0.1 | 0.1 | 0.1 |
| B | Orange oil | 0.45 | — | — |
| | Medium-chain fatty acid | 10 | — | — |
| | triglyceride | | | |
| C | Sucrose fatty acid ester | 0.355 | — | — |
| | Polyoxyethylene | 0.845 | — | — |
| | polyoxypropylene glycol | | | |
| D | Glycerin | 13.5 | 13.5 | 13.5 |
| E | I-carrageenan | 0.45 | 0.45 | — |
| | K-carrageenan | 0.2 | 0.2 | — |
| | Locust bean gum | 0.2 | 0.2 | — |
| | Sodium polyacrylate | 0.003 | 0.003 | — |
| F | Propyl parahydroxybenzoate | 0.015 | 0.015 | 0.015 |
| G | D-Sorbitol | 30 | 38 | 38 |
| J | Flavor | — | 0.1 | 0.1 |
| K | Purified water | 43.882 | proper | proper |
| | Total quantity | 100 | 100 | 100 |

Unit: g.

Test Method
16 healthy adult volunteers are given 3 g samples from test formulations (T-01(Practical example), T-02 (Comparative example) and T-03 (Comparative example)). T-02 is a gelled medicinal composition for oral administration containing no oil and no emulsion of the present invention. T-03 is a medical solution composition for oral administration containing no oil, no emulsion and no gelling agent of the present invention.

We evaluated the bitterness on a 5 grade system, as summarized in the table below.

| | Bitterness | | | | |
|---|---|---|---|---|---|
| | Not Bitter at | A | Somewhat | | |
| | all | little Bitter | Bitter | Bitter | Very Bitter |
| Score | 5 | 4 | 3 | 2 | 1 |

*Bitterness means bitterness itself and sensation tongue-biting.

Test Results

Bitterness

| Subject No. | T-01 | T-02 | T-03 |
|---|---|---|---|
| 1 | 5 | 4 | 3 |
| 2 | 5 | 4 | 4 |
| 3 | 4 | 4 | 4 |
| 4 | 2 | 4 | 3 |
| 5 | 3 | 4 | 3 |
| 6 | 2 | 2 | 2 |
| 7 | 5 | 4 | 3 |
| 8 | 5 | 3 | 2 |
| 9 | 4 | 4 | 3 |
| 10 | 4 | 3 | 3 |
| 11 | 4 | 4 | 2 |
| 12 | 4 | 4 | 3 |
| 13 | 3 | 5 | 2 |
| 14 | 4 | 3 | 2 |
| 15 | 4 | 4 | 4 |
| 16 | 5 | 2 | 3 |
| Total | 63 | 58 | 46 |
| Average | 3.94 | 3.63 | 2.88 |
| Deviation | 0.998 | 0.806 | 0.719 |

There was a significant difference between T-01 and T-02 (P < 0.01)

Discussion

The test results showed that T-01 (Practical example) score was 3.94, T-02(Comparative example) score was 3.63 and T-03(Comparative example) score was 2.88. The results demonstrate that the bitterness and tongue-biting sensation of donepezil, which is the base of the present invention, is remarkably masked. Since the present invention masks the unpleasant taste of the drug substance and it is a creamy gelled formulation that is easy for oral administration, it can be provided to patients suffering from senile dementia and Alzheimer's disease, to whom donepezil is administered for a long period of time.

Representative Manufacturing Protocol

The following provides a representative manufacturing protocol for gelled composition according to the present invention:

The 1^{st} process: Prepare necessary amount of water and polyol in a preparation tank.

The 2^{nd} process: Add donepezil, surfactants, preservatives, pH buffering agents in a tank while being heated.

The 3^{rd} process: Oils are added and stirred and emulsify in a tank.

The 4^{th} process: Gelling agents, thickening agents and sweetening agents are added to make the emulsion.

The 5^{th} process: Flavor is added and dispersed.

The 6^{th} process: The resultant composition is sterilized with heat.

The 7^{th} process: A disposable container is filled with resultant gelled composition and chilled it to make a product.

It is evident from the above discussion and results that the subject invention provides an important new donepezil gel composition. Advantages of the subject compositions include ease of ingestion by patients without experiencing an unpleasant taste, such as bitterness and paralyzing sensation. Furthermore, the compositions enhance the patient's visual awareness and attention by turning a clear water-soluble gelled composition of donepezil and/or its salts into a white emulsion. Finally, the compositions are well-suited for self-medication. As such, the subject invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising:
a donepezil active agent present in a gelled pharmaceutically acceptable vehicle comprising an emulsion of oil and water.

2. The composition according to claim 1, wherein said gelled pharmaceutically acceptable vehicle has a pH below about 8.

3. The composition according to claim 1, wherein said gelled pharmaceutically acceptable vehicle has a pH ranging from about 3 to about 7.

4. The composition according to claim 2, wherein said vehicle comprises an oil selected from the group consisting of: animal/vegetable oils, hardened animal/vegetable oils, fractionated animal/vegetable oils, fatty acid esters, tricaprin, cacao oil, cacao butter, orange oil, soybean phospholipids, palm oil, peanut oil, soybean oil, , cottonseed oil, and soybean oil mixture, eucalyptus oil, lavender oil, lemon oil, rose oil, mint oil, fennel oil, olive oil.

5. The composition according to claim 4, wherein said fatty acid esters are chosen from medium-chain fatty acid glyceride, isopropyl myristate, and octadecyl myristate,

6. The composition according to claim 2, wherein said oil is present in an amount ranging from about 0.1 to about 45 wt %.

7. The composition according to claim 2, wherein said gelled pharmaceutically acceptable vehicle comprises a surfactant.

8. The composition according to claim 7, wherein said surfactant is selected from the group consisting of potassium palmitate, sodium lauryl sulfate, potassium lauryl sulfate, polyoxyethylene alkylethers, polyoxyethylene alkenylethers, polyoxyethylene alkylphenylethers, polyoxyethylene hydrogenated castor oil, polyoxyethylene glycerin fatty acid ester, polyoxyethylene sorbitan fatty acid ester, glycerin fatty acid ester, sorbitan fatty acid ester, propylene glycol fatty acid ester, polyglycerin condensed ricinolein acid ester, polyglycerin fatty acid ester, lecithin, sucrose fatty acid ester and polyoxyethylene polyoxy propylene glycol.

9. The composition according to claim 8, wherein said surfactant is present in an amount ranging from about 0.05 to about 25 wt %.

10. The composition according to claim 2, wherein said gelled pharmaceutically acceptable vehicle further comprises a gelling agent.

11. The composition according to claim 10, wherein said gelling agent is present in an amount ranging from about 0.01 to about 10 wt %.

12. The composition according to claim 10, wherein said gelling agent is selected from the group consisting of: carrageenan, pectin, agar, alginic acid, sodium alginate, gelatin, mannan, konjak, konjak mannan, glucosic mannan, chitosan, acacia gum, xanthan gum, locust bean gum, tamarind seed polysaccharides, gellan gum, tragacanth gum, karaya gum, cassia gum, tara gum, guar gum, psyllium seed gum, gutti gum, and pullulan

13. The composition according to claim 12, wherein said composition further comprises a thickening agent.

14. The composition according to claim 13, wherein said thickening agent is present in an amount ranging from about 0.01 to about 10 wt %.

15. The composition according to claim 13, wherein said thickening agent is selected from the group consisting of: acacia gum, tragacanth gum, locust bean gum, dextrin, dextran, arabinogalactan, milk solid, pullulan, alginate propylene glycol ester, hydroxyethyl starch, carboxymethyl starch, hydroxypropyl starch copolydone, polyvinyl alcohol completely saponified, polyvinyl alcohol partially saponified, polyvinyl pyrrolidone, carboxyvinyl polymer, sodium polyacrylate, polyethylene glycolmacrogol, and cellulose or its derivatives such as cellulose gum, carmellose sodium, hydroxypropyl cellulose, hydroxyethyl methyl cellulose, methyl cellulose, carboxymethyl cellulose, copolyvidone, polyvinyl pyrrolidone, carboxyvinyl polymer, polyacrylic acid or partially neutralized polyacrylic acid, or salt and macrogol.

16. The composition according to claim 1, wherein said gelled pharmaceutically acceptable vehicle further comprises a polyol.

17. The composition according to claim 1, wherein said gelled pharmaceutically acceptable vehicle further comprises a sweetening agent.

18. The composition according to claim 1, wherein said gelled pharmaceutically acceptable vehicle further comprises a flavorant.

19. The composition according to claim 1, wherein said gelled pharmaceutically acceptable vehicle further comprises a preservative.

20. The composition according to claim 1, wherein said donepezil active agent is present in said gel composition in an amount ranging from about 0.0025 wt % to about 0.2 wt %.

21. A gelled donepezil composition comprising:
a donepezil active agent;
an emulsion of oil and water;
a surfactant;
a gelling agent; and
a thickening agent;
wherein said gelled composition has a pH ranging from about 3 to about 7.

22. A method of a making a gelled donepezil composition, said method comprising:
making an emulsion of water and oil; and
dissolving a donepezil active agent in said emulsion;
to make said gelled donepezil composition.

23. A packaged donepezil gelled composition comprising:
a single dosage of said donepezil gelled composition sealed in a disposable packaging element.

24. The packaged composition according to claim 23, wherein said disposable packaging element is a pouch.

25. A method of administering a donepezil active agent to a subject, said method comprising:
orally delivering a gelled donepezil composition according to claim 1 to a said subject to administer said donepezil active agent to a subject.

26. A kit comprising two or more individual dosages of a gelled donepezil composition according to claim 1.

27. The kit according to claim 26, wherein said two or more dosages are individually packaged.
